(19) United States
(12) Patent Application Publication
(10) Pub. No.: US 2015/0046465 A1
(43) Pub. Date: Feb. 12, 2015

(54) SYSTEM AND METHOD FOR TARGETING RELEVANT RESEARCH ACTIVITY IN RESPONSE TO DIAGNOSTIC MARKER ANALYSES

(71) Applicant: Rebecca Lambert, Washington, CT (US)

(72) Inventor: Rebecca Lambert, Washington, CT (US)

(21) Appl. No.: 14/524,550

(22) Filed: Oct. 27, 2014

Related U.S. Application Data
(63) Continuation-in-part of application No. 13/051,440, filed on Mar. 18, 2011, now Pat. No. 8,874,378.
(60) Provisional application No. 61/315,670, filed on Mar. 19, 2010.

(51) Int. Cl. G06F 19/00; G06F 17/30; G01N 33/53

(52) U.S. Cl. CPC G06F 19/366; G01N 33/53; G06F 17/30321; G01N 2800/22
USPC 707/741; 506/9; 506/7

(57) ABSTRACT

A system and method for targeting relevant research activity for clinical application in response to diagnostic markers analyses is described. Diagnostic analysis is performed to detect the level of each of at least three diagnostic markers. The levels of the tested markers are used to identify relevant publications from among a large database of articles. The most relevant literature, such as, one which reports research and studies that have been conducted to identify, moderate, and define the mechanisms unique to individual and combinations of diagnostic markers for various disease states, is then provided to the patient and/or the patient's physician, optionally with a summarization of the treatment recommendations from the provided literature. The customized information delivery provides a range of published peer-reviewed therapeutic options and/or published research studies.

### CROSS REFERENCE TO RELATED APPLICATIONS

This application is a continuation in part of U.S. patent application Ser. No. 13/051,440, filed Mar. 18, 2011, now U.S. Pat. No. 8,874,378, which claims priority under 35 U.S.C. §119(e) of U.S. provisional patent application No. 61/315,670, filed Mar. 19, 2010, entitled System and Method for Targeting Relevant Research Activity in Response to Angiogenic Regulator Analyses, the entire disclosures of which are hereby incorporated by reference.

### STATEMENT REGARDING FEDERALLY SPONSORED RESEARCH OR DEVELOPMENT

n/a

### BACKGROUND OF THE INVENTION

Monitoring diagnostic markers can help medical professionals discover important information about a patient's condition. These markers include various biologic regulators that can control various biologic functions, for example, hormones, metabolism, immunity and angiogenesis. As knowledge of how these regulators impact health continues to grow, it is difficult for medical professionals to be fully versed in all the literature which may be pertinent to a patient.

The diagnostic markers may have determined associations which can be applied to evaluate an individual's physiologic or disease state. Beyond biomarkers suggested to be direct or indirect protein regulators (e.g., such as those associated with angiogenesis), the biomarkers may also include diagnostic testing applied to tissues and other body fluids (e.g., urine or saliva) besides blood samples, and evaluation of cells (typing and enumeration). Such methods have been or may be identified for their association with disease development and diagnoses. Cellular profiling includes measures such as tissue and body fluid cell typing and quantitation (e.g., complete blood count; hematocrit; cell density; differentiation and antigen expression; and cell health status, such as apoptosis or apoptotic markers).

One example of the influences a diagnostic marker can have is the study of angiogenesis and angiogenic regulators. Angiogenesis refers to the growth of new blood vessels, either through sprouting or vessel splitting. It is a condition which exists in health on three occasions: menstruation, pregnancy and wound healing (when capillaries rush to a wound site to heal it and retreat after approximately 10-14 days). However, pathological angiogenesis, the abnormal and rapid development of blood vessels, is associated with and drives many diseases including cancer, psoriasis and age-related macular degeneration. In cancerous tissue, tumors cannot grow or spread (metastasize) without the development of new blood vessels. Blood vessels supply tissues with oxygen and nutrients necessary for survival and growth. Endothelial cells, the cells that form the walls of blood vessels, are the source of new blood vessels.

New vessel growth is tightly controlled by a finely tuned balance between factors that activate endothelial cell growth and those that inhibit it. Over 50 individual endogenous angiogenic regulators have been identified as angiogenic factors. They may be measured in blood, serum, urine, tissue and lymph samples. Regulators are broadly classified as: 1) angiogenic activators, stimulators, or growth factors; and 2) endogenous angiogenic inhibitors (cf. synthetic drugs intended to inhibit angiogenesis). It is the unique relationship between these two types of regulators that determines if angiogenesis occurs and thereby supports disease.

For example, about 50 proteins are currently known to regulate endothelial cell replication, some of which are shown in Table I.

TABLE I

| NAME | FULL NAME |
|---|---|
| VEG-F | Vascular Endothelial Growth Factor |
| EGF | Epidermal Growth Factor |
| bFGF-Basic | Fibroblast Growth Factor- Basic |
| IL-2 | Interleukin-2 |
| PDGF-BB | Platelet-derived Growth Factor-BB |
| TNF-α | Tumor Necrosis Factor- alpha |
| IL-1β | Interleukin-1 beta |
| IL-8 | Interleukin-8 |
| IL-10 | Interleukin-10 |
| TSP-1 | Thrombospondin-1 |
| COX-2 | Cyclooxygenace |
| HGF | Hepatocyte Growth Factor |
| IGF-1 | Insulin like Growth Factor |
| MMP-2 | Matrix Metalloproteinase-2 |
| MMP-9 | Matrix Metalloproteinase-9 |
| TNF-β | Tumor Necrosis Factor- beta |
| TGF-β | Transforming Growth Factor- beta |
| Angiogenin | Angiogenin |
| GM-CSF | Granulocyte Macrophage Colony-Stimulating Factor |
| Endostatin | Endostatin (collagen XVIII fragment) |
| Angiostatin | Angiostatin (plasminogen fragment) |
| IL-6 | Interleukin-6 |
| G-CSF | Granulocyte Colony-Stimulating Factor |
| IL-7 | Interleukin-7 |
| Kringle 5 | Kringle 5 (plasminogen fragment) |
| Angiopoitin-1 | Angiopoitin-1 |
| FGA/FGB | Fibrinogin |

At a critical point in the growth of a tumor, the tumor sends out signals to the nearby capillaries to activate new blood vessel growth. Two endothelial growth factors, VEGF and bFGF, are expressed by many tumors and seem to be among the most important biologic stimulators in sustaining tumor growth. A role of biologic inhibitors is to keep biologic stimulators within their normal range. Inhibitors have half lives which are measured in hours and days, while stimulators' half lives are considerably shorter, most measured in only minutes.

Although first discovered in the late 1960's at Harvard by Dr. Judah Folkman, angiogenesis as a field of knowledge and discovery is still largely dominated by research scientists. Although many physicians and some patients have heard the term angiogenesis, few understand the role of angiogenesis in cancer and numerous other diseases. Peer-reviewed literature documenting the role of angiogenesis in disease and in reversing disease, unless directly tied to a biologic inhibiting pharmaceutical, is customarily not read by most clinicians.

While studies documenting the beneficial effects of less than a dozen biologic inhibiting pharmaceuticals increase in number annually, during the last several decades many peer-reviewed studies have been published which demonstrate how beneficially moderating individual biologic inhibitors, through the utilization of natural compounds and other techniques, suppresses disease stimulating biologic growth factors. These studies are largely unknown to the practicing medical community. Studies have shown such natural compounds to be safe and effective in the treatment of disease, cost effective, and almost entirely without side effects, and yet most physicians have no knowledge of their well-documented benefit in treatment of disease.

It is estimated that the amount of published literature with respect to the various aspects of medicine, angiogenesis and the diseases it drives doubles every five years. This is but one example of a process with known diagnostic markers. Based solely on the volume of literature about diagnostic markers, the most advanced treatment options are not always known by practicing clinicians.

Additional cellular and molecular profiling of individuals and disease states includes a variety of methods applied to the evaluation of metabolic, immune system, hormonal, genetic, and epigenetic tests to determine a patient's disease status and assess the responses or impact of treatments. Some examples include:
Physical measures such as nuclear medical techniques, radiometric diagnostic technologies (e.g., X-rays, CT scans, and use of isotopic markers) and invasive procedures (e.g., biopsy, lumpectomy) used to monitor the patient's disease status (tumor size, type, viability, cellular markers/characteristics, and distribution) prior to and as a result of treatment regimes;
Metabolic status: Chemistry, biochemistry (reactive oxygen species, glutathione, fasting glucose, enzyme levels such as LDH, and enzyme activity), lipid composition (8-hydroxy deoxy guanosine, malondialdehyde, 4-hydroxyonenal) and metabolic hormones (insulin, proinsulin and GLP-1);
Inflammation or immune status (cytokines such as IL-2, IL-8, C-reactive protein, T cell proliferation and NK cell activity);
Hormones (e.g., thyroid and steroids) associated with patient and disease status;
Genetic markers identified by specific techniques (e.g., RFLP, SSLP, AFLP, microsatellite associated polymorphism) associated with specific diseases (e.g., BRCA1 and 2, and hereditary nonpolyposis colorectal cancer); and
Epigenetic markers (e.g., DNA and RNA methylation, histone modification) associated with over-expression of oncogenes, chromosomal instability, drug resistance and activation of latent viral genomes.

For a physician or patient to perform a literature search on the basis of a specific patient sample analysis to determine the full range of peer-reviewed medical articles providing relevant information with respect to the multitude of diagnostic markers would consume days if not weeks of extensive research. To date, it has not been known to couple the diagnostic markers with specific peer-reviewed studies providing a range of treatment options to thereby stimulate therapy discussions between physician and patient.

### BRIEF SUMMARY OF THE INVENTION

Disclosed is a system and method for targeting relevant research activity for clinical application in response to diagnostic marker analyses. A biologic analysis is performed on a patient, for example, on a blood sample in order to detect the level of various diagnostic markers, e.g., at least three in a first embodiment. While individual diagnostic markers have been correlated with a specific disease state or patient condition by researchers, the present approach precisely measures multiple markers simultaneously for diagnostic use by a clinician. The levels of the diagnostic marker are used as indexes to identify relevant peer-reviewed research publications from among a large database of articles. What are believed to be the most relevant peer-reviewed literature reporting research and studies that have been conducted to identify, moderate, and define the mechanisms unique to individual and combinations of diagnostic markers for various disease states are then provided to the patient and/or to the patient's physician.

In this manner, the vast amount of research-based information is winnowed down to a specifically relevant subset of literature and summarized detail and documents studies, such as to provide information for preventing disease capacity, for moderating the out-of-range marker(s) back to within normal or diagnostically relevant ranges, etc. This information in various formats to include lists, charts, spreadsheets, and all other means for organizing detailed, specific and variable information is then made available to the patient or the patient's physician for clinical therapy discussions and analysis. The customized information delivery provides the patient and physician a range of published peer-reviewed therapeutic options and/or published research studies, such as studies for moderating each of the out of range regulators to within normal or diagnostically relevant ranges. This unique matching of the literature to a patient's individual chemistry allows the practicing physician a broad range of options that have been evaluated by his peers with which to treat the patient's disease in a highly targeted and unique manner. The report according to the presently described innovation provides the physician with comprehensive research underpinning for the rationale and efficacy for treatment of abnormal markers by providing the full research documentation, optionally in addition to a summarization thereof for facilitating prescribing detail and dosing.

The biologic analysis may be performed by enzyme-linked immunosorbent assay (ELISA) techniques.

Once the patient (e.g., a sample from the patient) has been analyzed and levels of certain diagnostic markers have been obtained, this information is used to intelligently identify what are believed to be the most relevant peer-reviewed research literature pertaining to the respective markers. The articles or abstracts thereof may be provided to the patient and/or physician in hard copy by mail or courier services, or in electronic format via email, on a portable memory medium such a DVD or memory stick, or as a communications network address or link to each article or to a set of articles for the respective patient. If only article abstracts or other article identifiers are provided, the patient or physician may choose to obtain the entire article through one of various known literature sources.

Databases of peer-reviewed research studies, such as PUBMED, are well known in the art. In one embodiment of the present system and method, personnel perform an in-depth review of the literature relevant to each of a set of diagnostic markers. This pool of literature contains at a minimum articles that pertain to the diagnostic markers tested for in the analysis. The purpose of this in-depth literature review is to identify a number of research articles that appear to be, at the time of the in-depth review, the most relevant to the respective diagnostic markers, the relationship between the respective marker and various diseases and disease states, and to treatment options therefor. In one embodiment, these most-relevant articles represent a set of references that typically form part of the literature identified to the patient and/or physician each time the respective marker is tested for or each time the respective marker is found to have an abnormal or otherwise flagged level, e.g., in a patient sample.

However, additional patient information is used to further refine the set of literature identified to the patient and/or physician. For example, the patient sample may reflect a specific combination of markers having respective levels beyond a predetermined normal or diagnostically significant range. Such a combination may be used as the basis for a search engine query of the literature database for the purpose of locating relevant references that may not be part of the preferred set of literature. Since new research is documented at a rapid pace, literature that has been published subsequent to the previously described in-depth review can be discovered via the search engine query.

Additional diagnostic markers and/or patient information, such as suspected diagnoses, tumor state and location, family medical history, sex, age, allergies, etc., may also be used to form search engine queries to obtain additional relevant research literature. While algorithms can automatically assemble and execute the search engine queries into the literature database, it is expected that manual monitoring and adjustment of the query assembling algorithm may be performed for at least an initial time period to optimize the search results. For example, it may be found that search results having a date of publication within a certain period of time are more relevant due to some relatively recent discovery in the field; the search query would thus weight results within this time period more heavily. Manual intervention and monitoring of this search process has the added benefit of enabling the addition of newly found research literature to the preferred set of literature, and possibly the removal or substitution of older or now less favored articles from that preferred set.

Through these techniques, it becomes possible to filter the database contents according to a patient's biologic analysis. Only those studies that are relevant to the patient's condition are returned in a professionally informed priority manner.

Research literature relevant to moderation of diagnostic markers addresses a wide variety of pharmaceuticals, naturally occurring compounds, lifestyle choices (e.g. exercise), and integrative, complementary and alternative therapies that can be employed, much of which may be unknown to clinicians. For example, research has shown that certain pharmaceuticals have off-label applicability to the moderation of biologic stimulators. Thalidomide, now prescribed for leprosy and available off-label, is currently being used in clinical trials as a blocker of biologic growth factors (bFGF, VEGF, TNF-alpha). AVASTIN (Genentech, Inc.), currently prescribed for colorectal cancer, can be applied to the treatment of other cancers. Captopril, a high blood pressure medication, also has anti-angiogenic properties. Copper chelators, such as zinc, have been reported to impact diagnostic markers.

Studies have shown that numerous non-toxic compounds, in large measure spices, have beneficial impact on boosting inhibitors and repressing stimulators. For example, antioxidant N-acetyl-cysteine (NAC), which is available as an over-the-counter supplement, has been shown to create endothelial cell apoptosis and reduction of microvascular density within the core of the tumor. Further, curcumin has been identified as a substance that suppresses TNF-alpha, IL-1, and IL-6 biologic activators. In addition, studies may also indicate that certain lifestyle changes may be beneficial, including yoga, diet, sleep patterns, meditation and exercise.

The specific utility of the disclosed system and method lies in allowing clinicians and their patients to take advantage of the most advanced research for the specific determinants of the process (e.g., angiogenesis) which underlies the individual patient's disease.

### BRIEF DESCRIPTION OF THE SEVERAL VIEWS OF THE DRAWINGS

Other features and advantages of the present invention will be apparent from the following detailed description of the invention, taken in conjunction with the accompanying drawings of which:

FIG. 1 illustrates schematically a system for targeting relevant research activity in response to diagnostic marker analyses according to the present invention;

FIG. 2 illustrates an implementation of the general method of targeting relevant research activity in response to diagnostic marker analyses according to the present invention; and

FIG. 3 illustrates a further refinement of the method of FIG. 2.

### DETAILED DESCRIPTION OF THE INVENTION

The present invention pertains to systems and methods for targeting and summarizing relevant research activity in response to the analysis of diagnostic markers (such as, biologic regulators in a biological sample from a patient). In a first embodiment of the present invention, the levels of various diagnostic markers are measured. Alternative embodiments employ the analysis of at least three diagnostic markers, and more than ten diagnostic markers. These markers may be a single type of marker or a mix of markers, such as, hormonal regulators, angiogenic regulators, immune regulator, and/or metabolic regulators. Peer-reviewed research publications that pertain to the field of diagnosing and treating cancer and other diseases driven by imbalances in diagnostic markers are indexed according to the respective markers studied therein. The measured diagnostic markers levels are then used by a data processing unit to identify research studies that pertain to the moderation of the same subset of markers. The identified research is then summarized and provided to the patient and/or to the patient's treating physician.

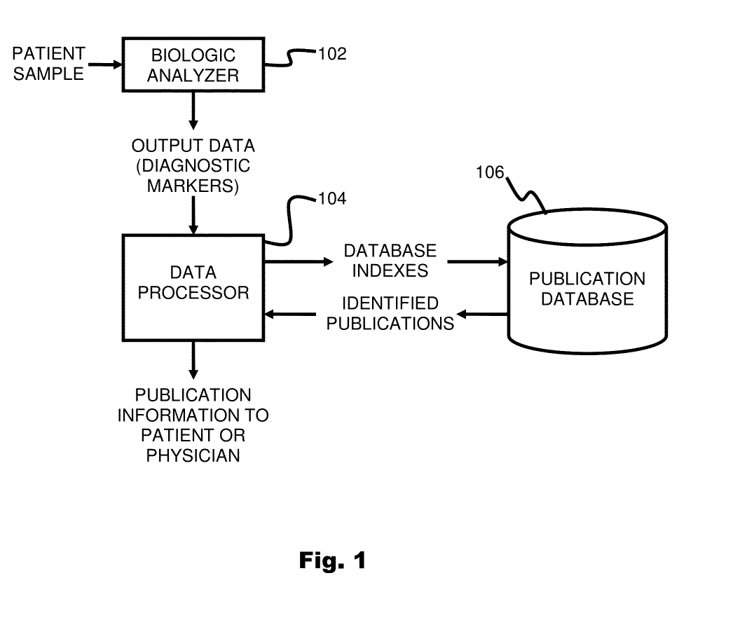

A blood sample can be drawn in any conventional manner. With respect to FIG. 1, the biologic analyzer 102 comprises test reagents and related equipment and may be performed using ELISA technology using sample processing, liquid handling and enzyme reading equipment. As is known, ELISA is a single-plex technology, meaning that for a given sample volume, only a single antigen can be quantitated at a time. This assay is based upon the specific recognition of the antigen of interest by antibodies that are bound to the surface of a micro titer well. These plate-bound antibodies capture their cognate antigen from complex biological samples. The resulting antigen-antibody complex is then detected by an enzyme-labeled antibody specific for the same antigen. Upon addition of the appropriate substrate, the enzyme produces a colorimetric reaction within the micro titer well in which the color density is directly proportional to the concentration of the protein within the sample. The Tecan FREEDOM EVOLYZER (Tecan Trading AG) is capable of fully automated ELISA processing and could be employed in the presently disclosed system and method.

Alternative embodiments of the present invention employ planar microarray technology or bead-based assays within the functional block labeled “biologic analyzer 102” in FIG. 1. The analysis can also be performed by magnetic particle assays like the Siemens Centaur system, fluorescent bead technology like the LUMINEX system (Luminex Corporation), chemiluminescence immunoassay technology like the Abbott Prism immunoassay analyzer, etc.

In one non-limiting embodiment, the panel of diagnostic markers that are tested in the biologic analyzer 102 include specific angiogenic regulators, such as, VEGF, EGF, bFGF-basic, IL-2, PDGF-BB but which may also include TNF-alpha, IL-1Beta, IL-8, IL-10, TSP-1, COX-2, HGF, IGF-1, MMP-2, MMP-9, TNF-Beta, TGF-Beta, Angiogenin, GM-CSF, Endostatin, Angiostatin, IL-6, G-CSF, IL-7, Angiopoitin-1 and all other angiogenesis growth factors, stimulators or endogenous inhibitors.

In another non-limiting embodiment the panel of diagnostic markers that are tested in the biologic analyzer 102 may include, in addition to the angiogenic regulators, metabolic status indicators (such as, reactive oxygen species, glutathione, fasting glucose, enzyme levels (e.g., LDH), and enzyme activity); lipid composition; cytokines (such as, IL-2, IL-8, C-reactive protein); hormones (such as, metabolic hormones (e.g., insulin, proinsulin and GLP-1), thyroid hormones, and steroids); genetic markers (such as, RFLP, SSLP, AFLP, microsatellite associated polymorphism); and epigenetic markers (DNA and RNA methylation, histone modification).

The results of the marker level analysis are stored in internal memory of the analyzer 102 and in a first embodiment are output in digital format to a portable data medium such as a DVD, memory stick, etc. Alternatively, the marker level analysis is transmitted from the analyzer as a sequence of electromagnetic signals over a transmission medium such as an electrically conductive or optical transmission line or over a wireless transmission medium via radiofrequency (RF), infrared (IR) or any other practical transmission means. In a further embodiment, the marker level analysis is output in printed (e.g., alphanumeric and/or graphical) form.

The measured marker levels are provided to a data processing unit 104, also referred to herein as a data processor, via a portable data medium interface such as a DVD reader, memory stick slot, etc., wireless or wired receiver, or keyboard, mouse, or touch-sensitive display screen in combination with a graphical user interface (GUI). Other conventional means for data input are used in further embodiments. The data processing unit implementing the methods of the present invention can be a standard personal computer, for example based upon an Intel or other microprocessor, including standard memory, disk drives and/or optical storage, data input/output facilities, network/communications interfaces, etc., or the data processing unit can be a customized data processor, especially configured to receive the output of the biologic analyzer and to use those data values in parsing a literature database Likewise, the data processing unit may be a general purpose processor running a software application (or ‘app’) which is configured to use the output of the biologic analyzer to search a literature database, as discussed in more detail below. The data processing unit may be embodied in a server, a desktop computer, a laptop computer, a tablet, a phone, a sample analysis instrument or any other device capable of performing data processing.

In a further embodiment, the biologic analyzer 102 and the data processor 104 may be provided within the same physical enclosure and therefore the distinction between the two would be functional rather than physical.

The data processor 104 is in communication with a publication database 106 which contains a large number of peer-reviewed research studies and publications, each of which in some fashion correlates the levels of certain diagnostic markers with a disease, a disease stage, a technique for moderating an imbalance among the respective diagnostic markers discussed therein to thereby treat or inhibit the growth of a disease or afflicting condition, modify diagnostic markers in general, or in a prevention of disease capacity, etc. For example, in one non-limiting embodiment, the publication database is the well-known PUBMED database, and the data processing unit is in communication therewith via a communications network such as the Internet.

In one embodiment, the publication database is parsed in order to identify each publication that pertains in a meaningful way with a diagnostic marker that is tested for by the biologic analyzer 102. A person or persons skilled in the art of biologic analysis and its role in cancer and other diseases reviews the results to identify a subset of publications that appear to be most relevant to one or more of disease diagnosis, treatment, biologic process moderation, etc. This subset forms a preferred set of literature that is typically summarized and reported to a patient and/or physician at least when the level of the respective marker is beyond a normal or diagnostically significant range.

Thus, if three diagnostic markers are included in the test panel performed by the biologic analyzer 102, the publication results provided to the patient and/or physician will include at least the preferred set of publications for each of the markers in the patient sample that were found to have a level beyond a respective normal or diagnostically significant range. In a further embodiment, the preferred set of publications for all of the markers in the patient sample that were tested in the analyzer is provided. Ideally, the steps involved in identifying the preferred set of literature for each marker are repeated at periodic intervals in order to ensure the latest relevant literature is included and added to the summary document.

In one non-limiting embodiment, the system and method of the present invention further includes the performance of an automated search on the basis of one or more of: the diagnostic markers in the patient sample that were tested and found to have a level beyond a predetermined normal or diagnostically significant range; all of the diagnostic markers tested by the analyzer; an actual patient disease diagnosis; a suspected patient disease diagnosis; a patient's family medical history; other characteristics of the patient's health including other diseases or conditions, allergies, etc. Literature from the database of peer-reviewed articles may be weighted by the query according to the date of publication—more recent publications may take precedence over later published studies. The results from the search query augment the relevant preferred set(s) of literature and summary treatment recommendations to be provided to the patient and/or physician.

It is recognized that such a query system, in order to be eventually automated, can be monitored and optimized by personnel skilled in the art of biologic research. During this learning period, the results of the query are monitored to assure that the most relevant literature is being returned and, if not, the content of the queries (e.g., how the queries are constructed) is modified. It is intended that the combination of the preferred set(s) of literature and the query results will ultimately provide the patient and/or physician with the most relevant and current research on biologic modulation, along with information in a summarized format on disease treatment and prevention.

The database 106 is a computer-readable memory configured with data for the practice of the methods of the present invention. The database can include main, or dynamic, memory that is directly accessible to a processor 104 and configured with publication data. It can also include optical, magnetic, fixed or removable media configured with publication and indexing data. The database may be hosted in a separate physical enclosure or may be provided on a memory facility associated with the data processor. It can also include a subscription-based, third-party hosted database accessible by a communications network such as the Internet. The data processor 104 uses the output data from the biologic analyzer as part of a search query into the publication database 106. Specifically, the data processor may be programmed with an identification of the preferred set of literature for each of the relevant diagnostic markers. This identification may be a pointer or address of a database entry, identification information that is adequate to allow the data processor to look up an article or research paper in the database, or an abstract of each of the documents making up the respective preferred set.

Alternatively, the sets of preferred literature may be stored in the publication database 106. In this case, the data processor uses the marker levels from the biologic analyzer 102 as indexes into the database to retrieve the preferred set(s) of literature as the “identified publications,” as illustrated in FIG. 1. In addition, as discussed above, the data processor performs one or more queries into the publication database on the basis of subsequent information provided by the patient or the patient's physician. The substance of this additional information is discussed more fully below.

In one non-limiting embodiment, the relevant peer-reviewed research studies are each correlated with a potential treatment option, which may include specific pharmaceutical therapies, chemotherapies, specific naturally occurring compound therapies, diet programs, exercise programs, or meditation-based therapies, all of which may be provided directly and/or in a summary format. Further, the relevant research studies may be pertinent to biologic modulation.

The matched research studies are then made available to the patient and/or the patient's physician so that the various techniques disclosed in the studies for marker moderation can be reviewed and discussed between the patient and physician for therapy planning and treatment decisions. This is of particular importance in the case of certain rapidly advancing diseases. The matched studies may be provided on a removable data carrier such as a DVD or memory stick, or as electronic data files that are stored at an addressable memory location on a communications network such as the World Wide Web or that are communicated to the patient or physician via electronic communications such as email. In any case, either the publication itself or a synopsis or abstract thereof may be provided as the output to the patient or physician.

The methods of the invention are implemented by computer program instructions which have been loaded into memory associated with the data processor 104. The program and the computer instructions comprising it can be introduced into the data processor then loaded into internal memory in any convenient manner, for example, by being read from removable optical or magnetic storage media on which it is recorded, or by being transmitted over network connections, etc. Once so introduced, the program instructions reside in the permanent storage of the data processor 104 until needed, whereupon they are loaded into dynamic memory accessible to the processor and cause the processor to perform the methods of the present invention.

The data processor 104 may be configured with such security measures as are known in the art to protect the privacy of individual patients in accordance with any applicable regulations.

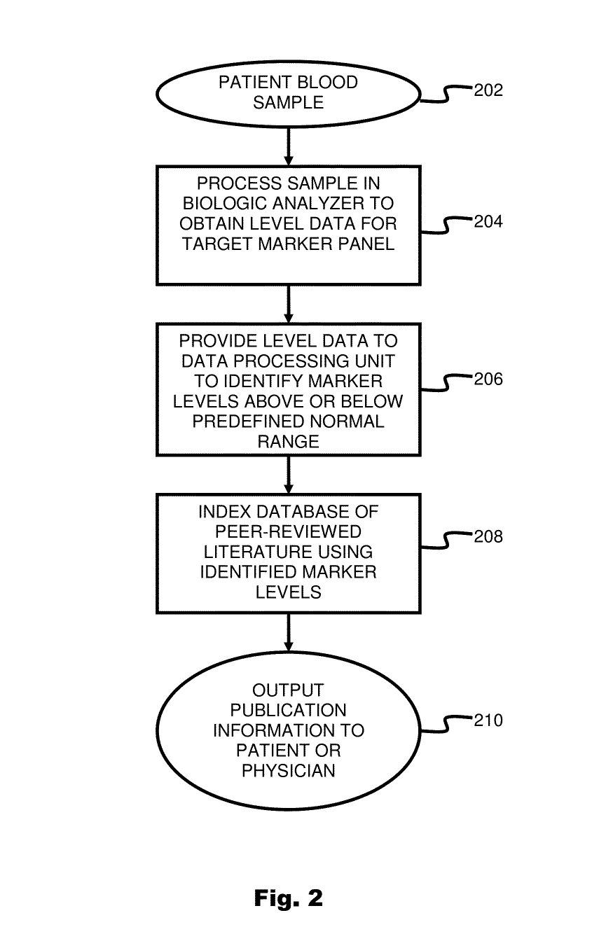

With regard to FIG. 2, a method of practicing one non-limiting embodiment of the present invention is disclosed. First, a patient blood sample is obtained 202 through techniques known in the art. The sample is then provided to an immunochemistry analyzer to obtain the level data for at least three target diagnostic markers (also referred to as a target marker panel) 204. Again, this is but one embodiment of the presently disclosed invention. Alternative embodiments employ five or more than ten target markers. Other embodiments may be performed by analyzing one or more samples. The samples may include samples other than blood samples, such as, tissue samples, saliva samples, urine samples, etc. The type of equipment that can be employed to carry out this step is described in greater detail in the foregoing.

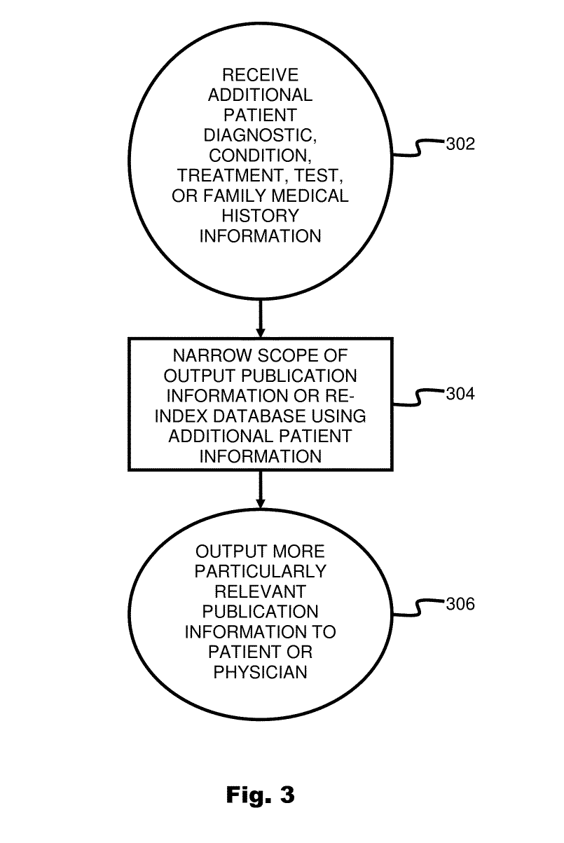

Once the level for each of the relevant diagnostic markers has been determined, they are provided to a data processing unit which can identify those markers, either stimulators or inhibitors, having a respective level that is above or below a predetermined normal range or other diagnostically determined range 206. The data processor then uses all of the measured marker level data, or alternatively just those marker levels outside the respective normal range or determined range, as indexes into a database of peer-reviewed literature 208. Such literature has been preprocessed to identify the respective diagnostic markers relevant thereto as well as the level ranges considered therein, all as discussed above. As in the foregoing, at least the preferred sets of literature (or identifiers thereof such as abstracts) for each of the tested markers having abnormal or other diagnostically significant level values, and optionally for all of the tested markers, are output to the patient and/or physician. Also as in the foregoing, this information may be augmented by the results of one or more queries performed by the data processor 104 on the contents of the publication database 106 using information obtained from the patient and/or physician. The latter concept is further discussed in the context of FIG. 3, below.

Finally, the research studies that correlate to the patient markers, level data, and optionally other patient data are output to the respective patient and/or his/her physician 210. This step may take many forms. For example, the recipient may be provided with an email including an electronic copy of each relevant study or an abstract thereof. Alternatively, the recipient may receive an email with network addresses, such as hypertext transfer protocol (HTTP) universal resource locators (URLs), which link to the relevant studies, or to abstracts thereof. Accompanying the identified publications, in one non-limiting embodiment, is a report of the patient diagnostic markers test results, optionally combined with a listing of the respective normal or diagnostically significant ranges. A summary of the additional patient information upon which the database was queried is also provided in an alternative embodiment.

With regard to FIG. 3, and as previously alluded to, a further embodiment of the presently disclosed system and method includes the ability to refine the set of peer-reviewed literature returned to the patient and/or clinician. Specifically, the data processor 104 is configured to receive additional data regarding the patient. This information may include a diagnosis or suspected diagnosis of a particular condition or disease or the determined stage of a disease, treatment that the patient has in the past undergone or is currently undergoing, other radiologic or biologic test results (such as Positron Emission Tomography (PET) scan Standardized Uptake Value (SUV) scores), a patient's mitotic count, current and prior medicines prescribed to the patient, patient allergies, and relevant aspects of the patient's family medical history, among other factors. The provision of this information may be prompted by the data processor 104 providing an on-line survey to the patient or to the patient's physician, whereby the respondent (the patient and/or physician) goes through a series of inquiries requesting the additional information. The inquiries may ask for size and quantity of tumors, primary tumor site, time frame since last scan, and other assorted information. Alternatively, the respondent may choose from a list of additional information that is useful to be provided. In another embodiment, the system of FIG. 1 and the method of FIG. 2 include a service representative who asks the respondent for the additional information and who inputs this information to the data processor.

Once the additional information has been provided, the data processor uses customized algorithms or artificial intelligence to parse the data on the basis of key words or via pattern matching. The additional information is then used to form queries that are input to a search engine operating on the content of the publication database 106. The output literature may be organized according to publication date, whether each pertains to treatment versus diagnosis, relevance on the basis of the number of diagnostic markers considered therein that are in common with those of the patient sample having abnormal levels, etc. This enables the more rapid identification of potentially relevant studies by the patient and/or physician.

In yet a further embodiment of the present inventor, the system and method provide a feedback path from the patient and/or physician so that individual results can be graded or otherwise scored as to their relevance to the patient's situation. Such feedback can for instance be used to adjust the content of the preferred set of literature for one or more markers, or to fine tune the query process that employs patient-specific information as input and adjust summary recommendations from the content of the preferred set of literature.

By connecting research dots (e.g., peer-reviewed studies) to a patient's unique chemistry, it provides the physician and the patient peer-reviewed research with summary treatment recommendation to discuss therapy options. For many diseases driven by biological processes, of which cancer and coronary artery disease are but two examples, time is often critical to the survival of the patient. One of the benefits of various embodiments of the inventor is that precious time is saved in the treatment of the disease by allowing the clinician to precisely target the underlying drivers of the patient's disease on the basis of the most current research treatment.

Many changes in the details, materials, and arrangement of parts and steps, herein described and illustrated, can be made by those skilled in the art in light of teachings contained hereinabove. Accordingly, it will be understood that the following claims are not to be limited to the embodiments disclosed herein and can include practices other than those specifically described, and are to be interpreted as broadly as allowed under the law.

What is claimed is:

1. A system for targeting relevant research activity in response to diagnostic marker analyses, comprising:
a biologic analyzer configured to receive a patient sample, to measure in the patient sample a level of each of at least three diagnostic markers, and to generate a data output indicative of the respective level of each of the at least three measured diagnostic markers;
a database unit comprising a plurality of entries, each entry corresponding to a respective publication and indexed at least according to a range of levels for each of at least one diagnostic marker; and
a data processing unit in communication with the biologic analyzer and the database unit and configured to receive the data output from the biologic analyzer, to identify entries in the database unit for which one or more of the patient diagnostic marker levels are addressed therein, and to provide an identification of the identified database entries.

2. The system of claim 1, wherein the at least three diagnostic markers include at least three of: hormonal regulators, angiogenic regulators, immune regulators, and metabolic regulators.

3. The system of claim 1, wherein the at least three diagnostic markers are selected from the group consisting of Vascular Endothelial Growth Factor, Epidermal Growth Factor, Fibroblast Growth Factor-Basic, Interleukin-2, Platelet-derived Growth Factor-BB, Tumor Necrosis Factor-alpha, Interleukin-1 beta, Interleukin-8, Interleukin-10, Thrombospondin-1, Cyclooxygenace, Hepatocyte Growth Factor, Insulin like Growth Factor, Matrix Metalloproteinase-2, Matrix Metalloproteinase-9, Tumor Necrosis Factor-beta, Transforming Growth Factor-beta, Angiogenin, Granulocyte Macrophage Colony-Stimulating Factor, Endostatin (collagen XVIII fragment), Angiostatin (plasminogen fragment), Interleukin-6, Granulocyte Colony-Stimulating Factor, Interleukin-7, Kringle 5 (plasminogen fragment), Angiopoitin-1, and Fibrinogin.

4. The system of claim 1, wherein the biologic analyzer measures the level of each of the at least three diagnostic markers via a technique selected from the group consisting of enzyme-linked immunosorbent assay (ELISA), planar microarray, bead-based assays, magnetic particle assays, and chemiluminescence immunoassays.

5. The system of claim 1, wherein the database unit further comprises, in conjunction with each of the plurality of entries, relevant data of the respective publication, the relevant data comprising at least one of: text, data and images.

6. The system of claim 5, wherein the data processing unit is further configured to provide, in conjunction with the identification of the identified database entries, the relevant data of each of the respective publications.

7. The system of claim 1, wherein each identification of the identified database entries is provided as an address of the respective publication in a database of publications accessible by a communications network.

8. The system of claim 1, wherein each publication is a peer-reviewed research study from which the level of at least one diagnostic marker is correlated with a recommended treatment option.

9. The system of claim 8, wherein the recommended treatment regime is selected from the group consisting of specific pharmaceutical, chemo- or radiation therapies, specific naturally occurring compound therapies, diet programs, exercise programs, meditation-based therapies, and integrative, complementary , and alternative therapies.

10. The system of claim 8, wherein the recommended treatment regime is for the purpose of biologic process inhibition.

11. The system of claim 1, wherein the data processing unit is further configured to receive additional information and to identify from among the database unit entries for which the patient additional information is relevant.

12. The system of claim 11, wherein the additional information is selected from the group consisting of additional diagnostic markers, a diagnosed patient condition, a suspected patient condition, a diagnosed disease stage, mitotic count, radiologic or biologic test results, current and prior medications prescribed, allergies, and family medical history.

13. The system of claim 1, wherein the data processing unit is further configured to extract treatment recommendations from the identified database entries and to provide a summarization of the treatment recommendations in conjunction with the identification of the identified database entries.

14. A method of targeting relevant research activity in response to diagnostic markers analyses, comprising:
receiving data indicative of a respective level of each of at least three measured diagnostic markers;
accessing a database unit comprising a plurality of entries, each entry corresponding to a respective publication and each entry is indexed at least according to a range of levels for each of at least one diagnostic marker;
identifying, with a data processor, a subset of database entries from the plurality of entries in the database unit for which the patient diagnostic marker levels in the data are within the respective ranges of levels; and
providing an identification of the identified subset of database entries.

15. The method of claim 14, wherein the at least three diagnostic markers include at least three of: hormonal regulators, angiogenic regulators, immune regulators, and metabolic regulators.

16. The method of claim 14, wherein the at least three diagnostic markers are selected from the group consisting of Vascular Endothelial Growth Factor, Epidermal Growth Factor, Fibroblast Growth Factor-Basic, Interleukin-2, Platelet-derived Growth Factor-BB, Tumor Necrosis Factor-alpha, Interleukin-1 beta, Interleukin-8, Interleukin-10, Thrombospondin-1, Cyclooxygenace, Hepatocyte Growth Factor, Insulin like Growth Factor, Matrix Metalloproteinase-2, Matrix Metalloproteinase-9, Tumor Necrosis Factor-beta, Transforming Growth Factor-beta, Angiogenin, Granulocyte Macrophage Colony-Stimulating Factor, Endostatin (collagen XVIII fragment), Angiostatin (plasminogen fragment), Interleukin-6, Granulocyte Colony-Stimulating Factor, Interleukin-7, Kringle 5 (plasminogen fragment), Angiopoitin-1, and Fibrinogin.

17. The method of claim 14, wherein the data indicative of a respective level of each of at least three measured diagnostic markers is received from a biologic analyzer that measures the level of each of the at least three diagnostic markers via a technique selected from the group consisting of enzyme-linked immunosorbent assay (ELISA), planar microarray, bead-based assays, magnetic particle assays, and chemiluminescence immunoassays.

18. The method of claim 14, wherein the wherein the data indicative of a respective level of each of at least three measured diagnostic markers is received from a biologic analyzer that measures the level of each of the at least three diagnostic markers in at least one of: a blood sample, a tissue sample, a urine sample, and a saliva sample.

19. The method of claim 14, wherein the database unit comprises, in conjunction with each of the plural entries, relevant data of the respective publication, the relevant data comprising at least one of: text, data and images.

20. The method of claim 18, wherein the database unit is further configured to provide, in conjunction with the identification of the identified database entries, the relevant data of each of the respective publications.

21. The method of claim 14, wherein the database unit is further configured to provide, in conjunction with each of the plurality of entries, an address of the respective publication in a database of publications accessible by a communications network.

22. The method of claim 14, wherein the database unit is further configured to correspond each of the plural entries with a respective peer-reviewed research study from which levels of at least one diagnostic marker is correlated with a recommended treatment option.

23. The method of claim 21, wherein the recommended treatment regime is selected from the group consisting of specific pharmaceutical, chemo- or radiation therapies; specific naturally occurring compound therapies; diet programs; exercise programs; meditation-based therapies; and
integrative, complementary, and alternative therapies.

24. The method of claim 14, wherein the recommended treatment regime is for the purpose of biologic process inhibition.

25. The method of claim 14, further comprising receiving additional patient information and identifying from among the identified database unit entries for which the additional patient information is relevant.

26. The method of claim 25, wherein the additional patient information is selected from the group consisting of additional diagnostic markers; a diagnosed patient condition; a suspected patient condition; a diagnosed disease stage; mitotic count; radiologic or biologic test results;
current and prior medications prescribed; allergies; and family medical history.

27. The method of claim 14, further comprising extracting treatment recommendations from the identified database entries and providing a summarization of the treatment recommendations in conjunction with the identification of the identified database entries.

28. The method of claim 14, wherein the data processor is embodied in a mobile device.
